(19) United States
(12) Patent Application Publication
(10) Pub. No.: US 2018/0137257 A1
(43) Pub. Date: May 17, 2018

(54) SENSING DEVICE, SENSING METHOD, AND SENSING PROGRAM

(71) Applicant: National Institute of Information and Communications Technology, Koganei-shi, Tokyo (JP)

(72) Inventors: Hiroto Tanaka, Koganei-shi (JP); Hiroaki Kojima, Koganei-shi (JP); Yasushi Naruse, Koganei-shi (JP); Masato Okada, Kashiwa-shi (JP)

(21) Appl. No.: 15/573,089

(22) PCT Filed: May 10, 2016
(86) PCT No.: PCT/JP2016/063893

§ 371 Date: Nov. 9, 2017

(30) Foreign Application Priority Data
May 11, 2015 (JP) 2015-096825

(51) Int. Cl. G06F 19/00; C12Q 1/02; G06F 17/18

(52) U.S. Cl. CPC G06F 19/707; G06F 17/18; C12Q 1/025; G06F 19/705

(57) ABSTRACT

In order to provide a sensing device and the like capable of using the behavior of a plurality of microorganisms changing over time as a sensor for estimating a chemical substance, the present invention includes: a recording unit in which is recorded a plurality of sample data that statistically calculated in advance the behavior of a plurality of microorganisms changing over time in an environment with known chemical substances present; a statistics unit for generating statistical data that statistically calculated the behavior of the plurality of microorganisms changing over time in an environment with a predetermined chemical substance present; and a determination unit for estimating the predetermined chemical substance on the basis of the statistical data and the sample data.

### TECHNICAL FIELD

The present invention relates to a sensing device and the like that use the behavior of a plurality of microorganisms as a sensor.

### TECHNICAL BACKGROUND

Living organisms recognize their environment using particular specialized senses from among the five senses; for example, dogs use their sense of smell, bats use their sense of hearing, and nematodes use their sense of taste. Biosensors in which the superior senses possessed by these organisms are utilized have attracted attention in recent years in the fields of medicine, science, and industry. Patent document 1, for example, provides an example of a biosensor of this type.

### DOCUMENTS OF THE PRIOR ART

### Patent Documents

Patent document 1: Japanese Unexamined Patent Application (JP-A) No. 2013-246112

### DISCLOSURE OF THE INVENTION

### Problems to be Solved by the Invention

However, because the aforementioned biosensors convert the specialized senses of an organism into some type of physical quantity, they have the drawback that they are not able to identify or quantify chemical substances. The further drawback exists that it is difficult for specialized senses of an organism that cannot be converted into physical quantities to be used as a biosensor.

For this reason, as a result of repeated strenuous investigations, the inventors submitting the present application discovered that, by using the behavior of microorganisms that have been placed in an environment in which a predetermined chemical substance is present as a sensor, then if this behavior is converted into statistical data and then used, it is possible to identify and quantify the aforementioned predetermined chemical substance.

The invention of the present application was achieved for the first time after the above-described discovery was attained, and it is a principal object thereof to provide a sensing device that, by using the behavior of a plurality of microorganisms that change over time as a sensor, is able to evaluate and quantify a chemical substance.

### Means for Solving the Problem

The sensing device of the present invention is characterized by being provided with a recording part in which are recorded a plurality of sample data items created by compiling in advance in statistical form the behavior of a plurality of microorganisms that change over time in an environment in which a known chemical substance is present, a statistical part that creates statistical data by compiling in statistical form the behavior of the plurality of microorganisms that change over time in an environment in which a predetermined chemical substance is present, and a determination part that, using the statistical data and the sample data, estimates the predetermined chemical substance.

If this type of structure is employed, then by acquiring for each chemical substance and for each chemical substance concentration a plurality of sample data items created by compiling in advance in statistical form the behavior of a plurality of microorganisms in an environment in which a known chemical substance is present, and by using this sample data together with statistical data created by compiling in statistical form the behavior of a plurality of microorganisms placed in an environment in which a blind (unknown) chemical substance is present, it becomes possible to estimate the blind chemical substance, and estimate the concentration of the chemical substance. Moreover, even if the specialized senses of an organism are not able to be converted into physical quantities, by compiling in statistical form the behavior of an organism that is converted over time, that organism can be used as a biosensor.

An example of a specific aspect of the above-described sensing device is one in which the determination part creates a model based on the sample data, and uses Bayesian inference to estimate the predetermined chemical substance by adapting the statistical data to the model.

If this type of structure is employed, then because the determination part estimates the predetermined chemical substance using Bayesian inference, even if the model created based on the sample data is a complex one, operating the model can be simplified compared to when a maximum likelihood method is used.

An example of a specific aspect of the above-described sensing device is one in which the statistical part creates the statistical data using ensemble averages.

If this type of structure is employed, then as a result of the statistical part using ensemble averages, because it is possible to compile the behavior of the microorganisms in statistical form while considering irregularities and the like caused by individual differences in the microorganisms, the accuracy of the statistical data can be further improved.

An example of a specific aspect of the above-described sensing device is one in which the microorganisms are bacteria, and the behavior of the bacteria is a rotational behavior.

If this type of structure is employed, then because the microorganisms are bacteria, handling them is easy, and if, for example, Escherichia coli (E. coli) bacteria are used as the bacteria, then these can be easily cultured. As a consequence, acquiring statistical data can be easily achieved. Moreover, if the E. coli bacteria are genetically engineered so as to create bacteria whose taste senses prefer a specific chemical substance, then it is possible to increase the number of categories of chemical substances that can be measured.

Moreover, a sensing method and a sensing program that are characterized in that they estimate a predetermined chemical substance using sample data created by compiling in advance in statistical form the behavior of a plurality of microorganisms that change over time in an environment in which a known chemical substance is present, and statistical data created by compiling in statistical form the behavior of the plurality of microorganisms that change over time in an environment in which the predetermined chemical substance is present, are also aspects of the present invention.

### Effects of the Invention

According to the present invention, it is possible to provide a sensing device and the like that are able to estimate a chemical substance by using the behavior of a plurality of microorganisms that change over time as a sensor.

### BRIEF DESCRIPTION OF THE DRAWINGS

FIG. 1 is a schematic view showing a sensing device according to the present embodiment.

FIG. 2 is a circuit diagram showing the sensing device according to the present embodiment.

FIGS. 3(a), 3(b), 3(c), and 3(d) are graphs showing sample data of the present embodiment.

FIGS. 4(a), 4(b), 4(c), and 4(d) are graphs showing sample data of the present embodiment.

FIGS. 5(a), 5(b), 5(c), and 5(d) are graphs showing sample data of the present embodiment.

FIG. 6 is a graph showing 11 parameters in one sample data item of the present embodiment.

FIG. 7 is a graph created by a determination part of the present embodiment.

### DESCRIPTION OF REFERENCE CHARACTERS

1 . . . Sensing device

2 . . . Statistical part

3 . . . Recording part

4 . . . Determination part

### BEST EMBODIMENTS FOR IMPLEMENTING THE INVENTION

An embodiment of the sensing device of the present invention will now be described with reference made to the drawings.

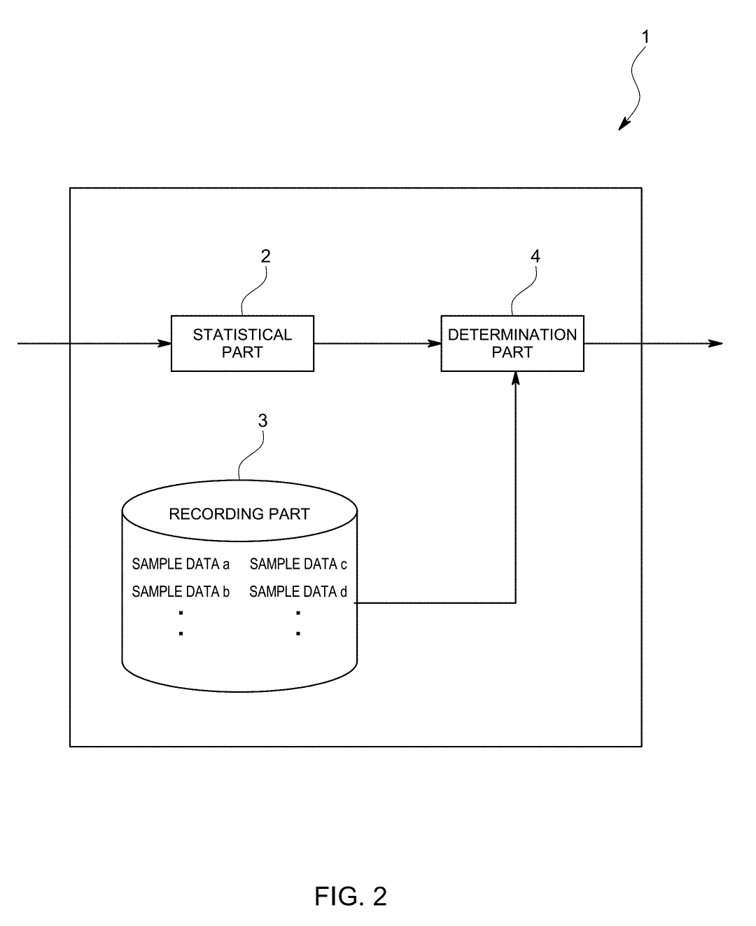

A sensing device 1 of the present invention is a sensing device 1 that analyzes chemical substances and, as is shown in FIG. 2, is provided with a statistical part 2 that creates statistical data by compiling in statistical form the behavior of a plurality of microorganisms that change over time in an environment in which a predetermined chemical substance is present, a recording part 3 in which are recorded a plurality of sample data items created by compiling in advance in statistical form the behavior of a plurality of microorganisms that change over time in an environment in which a known chemical substance is present, and a determination part 4 that, using the statistical data and the sample data, determines the predetermined chemical substance. Note that the term ‘estimates a chemical substance’ refers to either estimating a chemical substance or estimating the concentration of a chemical substance.

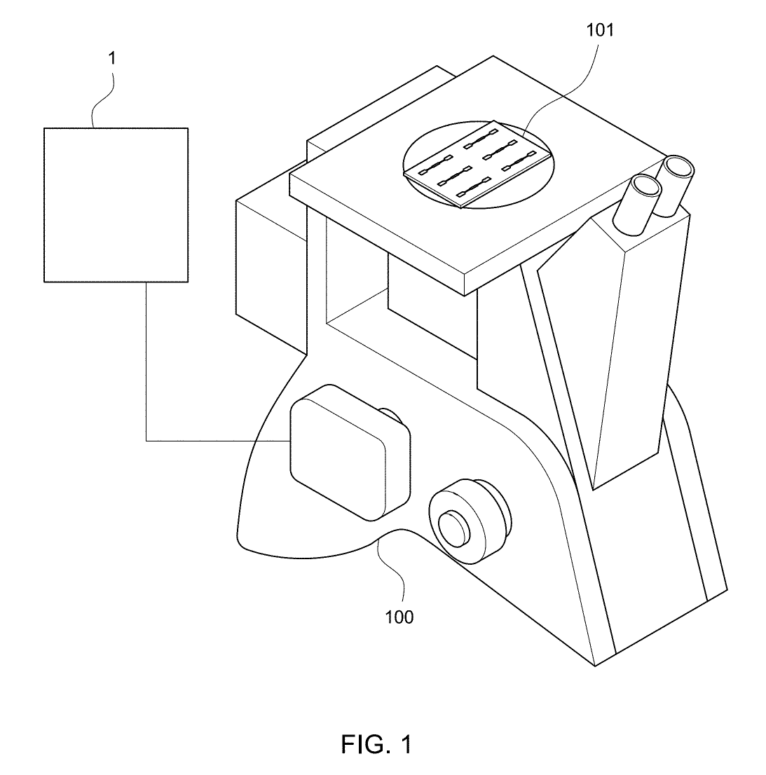

The statistical part 2 creates statistical data by compiling in statistical form the behavior of a plurality of microorganisms which change over time in an environment in which a predetermined chemical substance is present. In the present embodiment, moving image data is obtained by acquiring images of a state in which a sample is introduced into a flow path 101 to which microorganisms have been adhered, and statistical data is then created from this moving image data. As is shown in FIG. 1, for example, a CCD camera or a CMOS camera mounted on an inverted microscope can be used as an image acquisition device 100 that acquires these moving images. Note that the image acquisition device 100 is not limited to these, and may also be formed, for example, by a CCD camera or a CMOS camera that is mounted on an ocular lens of a microscope.

Here, the aforementioned state in which a sample is introduced into the flow path 101 to which microorganisms have been adhered refers to a state at the point in time when a sample is introduced into the flow path 101, or a state immediately after the introduction of a sample into the flow path 101 has been completed. An example of the period for which the image acquisition device 100 performs the image acquisition is a period lasting until the flow rate of the sample immediately after it has been introduced into the flow path 101 gradually decreases and reaches a particular fixed flow rate, however, this image acquisition period can be suitably altered to suit the microorganism.

The statistical part 2 separates the behavior of the microorganisms into a plurality of patterns, and calculates which pattern the behaviors of a plurality of microorganisms filmed in a predetermined unit area out of the plurality of microorganisms contained in the video data can be adapted to in a specific period in the form of statistical data. Specifically, the statistical part 2 performs the above-described statistical calculation using ensemble averages.

In the present embodiment, E. coli bacteria are used as the microorganisms, and these E. coli bacteria have chemotaxis that causes them to make directional movements relative to the concentration gradient of a specific chemical substance. Namely, these E. coli bacteria have properties that cause them to rotate counterclockwise in the case of a preferable substance (i.e., an attractant substance), and cause them to rotate clockwise in the case of a non-preferable substance (i.e., a repellent substance). Therefore, in the statistical part 2, the behavior of the E. coli bacteria is separated into three patterns, namely, a clockwise rotation state, a counterclockwise rotation state, and a stationary state. At a specific point in time, of all the E. coli bacteria contained within a predetermined unit area, the proportion of E. coli bacteria rotating clockwise, the proportion of E. coli bacteria rotating counterclockwise, and the proportion of E. coli bacteria that are in a stationary state are calculated respectively using ensemble averages, and these are then compiled in statistical form in predetermined time intervals so as to create statistical data. This statistical data is then transmitted to the determination part 4.

The recording part 3 records sample data created by compiling in advance in statistical form the behavior of a plurality of microorganisms that change over time in an environment in which a known chemical substance is present, and records a plurality of sample data items that are created for each category of chemical substance and each concentration of a chemical substance.

In the same way as the statistical data created by the statistical part 2, in this sample data the behavior of the microorganisms is separated into a plurality of patterns, and which pattern the behaviors of a plurality of microorganisms filmed in a predetermined unit area out of the plurality of microorganisms contained in the video data can be adapted to in a specific period is calculated in advance in the form of statistical data. Note that in the present embodiment, E. coli bacteria are used for the sample data in the same way as for the statistical data, and the behavior of these E. coli bacteria is separated into three patterns, namely, a clockwise rotation state, a counterclockwise rotation state, and a stationary state. At a specific point in time, of all the E. coli bacteria contained within a predetermined unit area, the proportion of E. coli bacteria rotating clockwise, the proportion of E. coli bacteria rotating counterclockwise, and the proportion of E. coli bacteria that are in a stationary state are calculated respectively using ensemble averages, and these are compiled in advance in statistical form in predetermined time intervals.

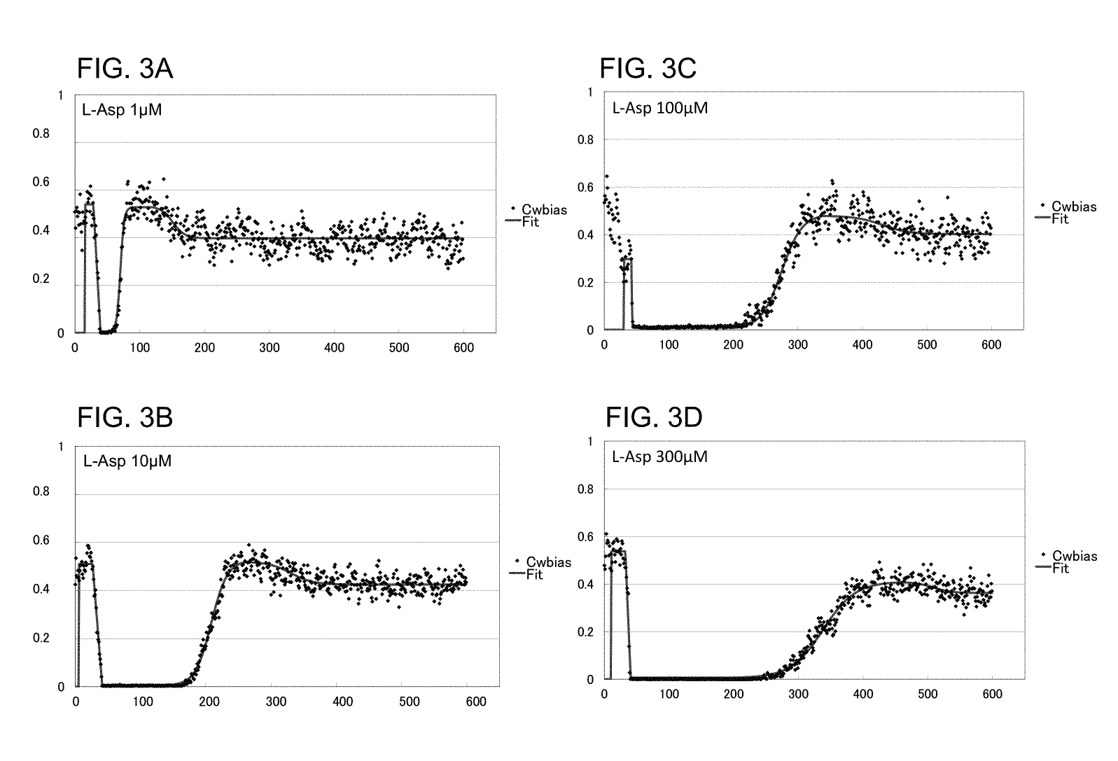

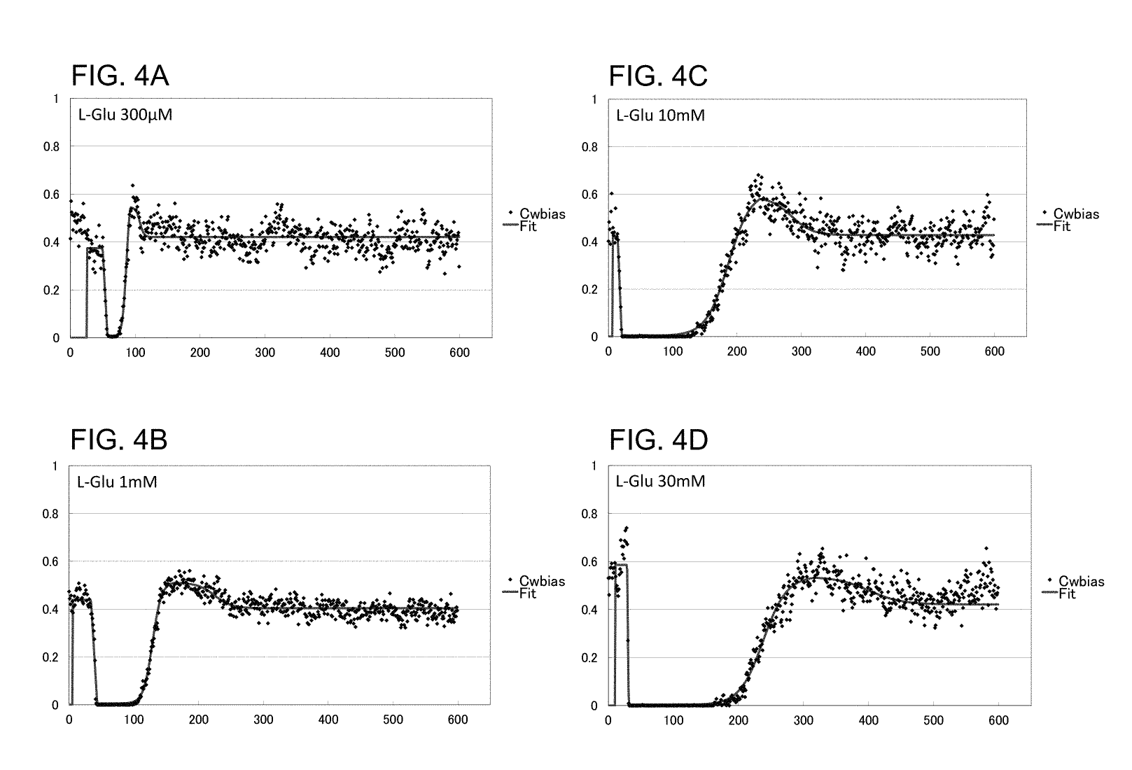

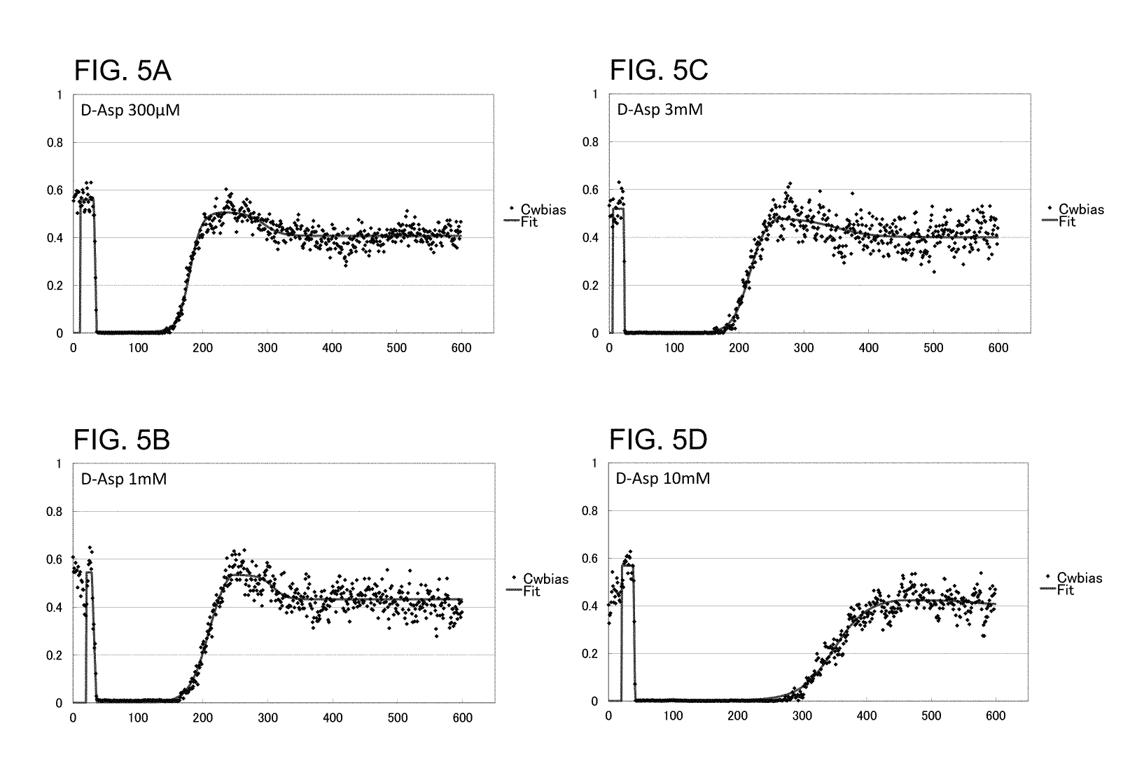

Examples of this sample data are shown in graph form in FIG. 3, FIG. 4, and FIG. 5. Note that in these graphs, the vertical axis shows the proportion of microorganisms that exhibit clockwise rotation behavior out of all of the E. coli bacteria contained within a unit area, while the horizontal axis shows time(s).

FIG. 3 shows in graph form sample data created by compiling in statistical form the behavior of microorganisms that change over time when L-aspartic acid (abbreviated as L-Asp in FIG. 3), as represented by the following chemical formula, is introduced as a sample into the flow path 101. Moreover, the concentrations of the respective L-aspartic acids are mutually different in each of (a), (b), (c), and (d) in FIG. 3, and are set to 1 μM in (a), 10 μM in (b), 100 μM in (c), and 300 μM in (d).

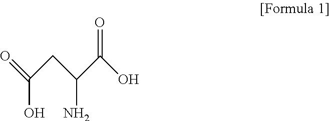

FIG. 4 shows in graph form sample data created by compiling in statistical form the behavior of microorganisms that change over time when L-glutamic acid (abbreviated as L-Glu in FIG. 4), as represented by the following chemical formula, is introduced as a sample into the flow path 101. Moreover, the concentrations of the respective L-glutamic acids are mutually different in each of (a), (b), (c), and (d) in FIG. 4, and are set to 300 μM in (a), 1 mM in (b), 10 mM in (c), and 30 mM in (d).

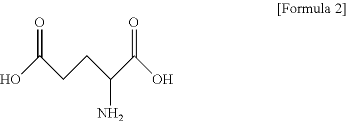

FIG. 5 shows in graph form sample data created by compiling in statistical form the behavior of microorganisms that change over time when D-aspartic acid (abbreviated as D-Asp in FIG. 5), as represented by the following chemical formula, is introduced as a sample into the flow path 101. Moreover, the concentrations of the respective D-aspartic acids are mutually different in each of (a), (b), (c), and (d) in FIG. 5, and are set to 300 μM in (a), 1 mM in (b), 3 mM in (c), and 10 mM in (d).

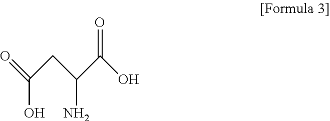

As can be seen from FIG. 3, FIG. 4, and FIG. 5, when the sample data is exhibited in graph form, the waveforms thereof vary slightly depending on the category and concentration of the samples.

The determination part 4 uses the statistical data created by the statistical part 2 and the sample data recorded by the recording part 3 to estimate the predetermined chemical substance used when the statistical data was being acquired. Specifically, the determination part 4 acquires sample data from the recording part 3 and creates a model based on this sample data. The determination part 4 then estimates the predetermined chemical substance using Bayesian inference to adapt the statistical data to the model.

This method of analysis is described below in detail.

Firstly, the determination part represents the sample data recorded by the recording part 3 in a graph whose vertical axis shows the proportion of microorganisms that exhibit clockwise rotation behavior out of all of the E. coli bacteria contained within a unit area, and whose horizontal axis shows time, and then extracts values based on 11 parameters from this graph.

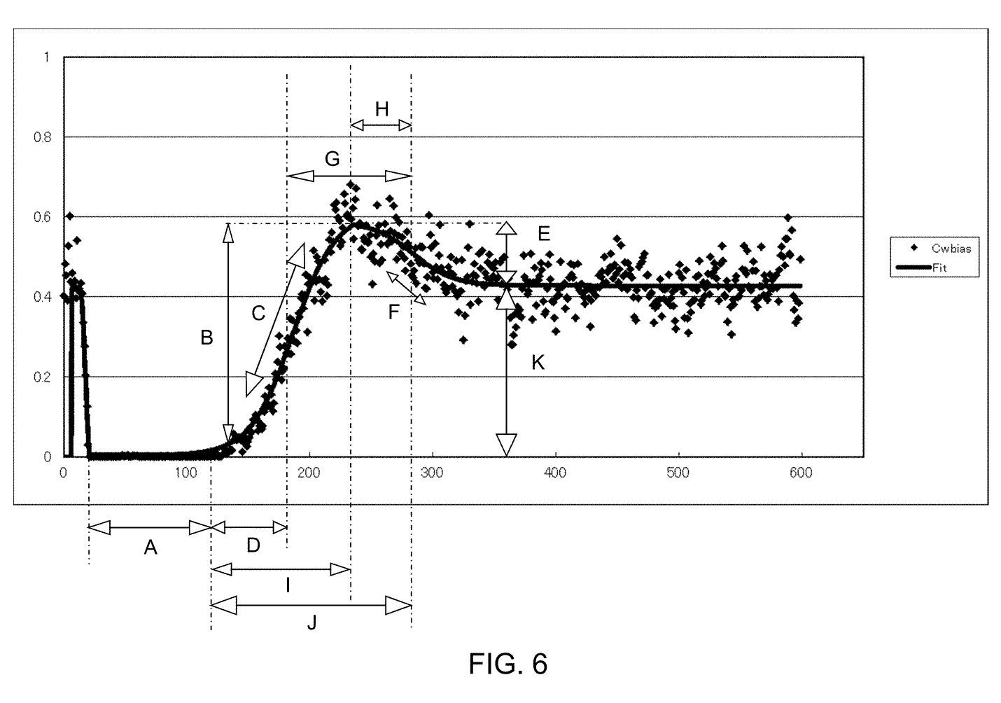

As is shown in FIG. 6, these 11 parameters are as follows.

(1) A time interval when the proportion of microorganisms rotating clockwise is 0%.

(2) An amplitude from a rise position when the proportion of microorganisms rotating clockwise begins to rise on a positive slope from 0% to a peak to which the proportion rises on this positive slope.

(3) The positive slope.

(4) A time interval from the rise position to halfway to the peak.

(5) An amplitude from the peak to a fall position to which the proportion falls on a negative slope.

(6) The negative slope.

(7) A time interval from the center of the positive slope to the center of the negative slope.

(8) A time sense from the peak to the center of the negative slope.

(9) A time interval from the rise position to the peak.

(10) A time interval from the rise position to the center of the negative slope.

(11) An amplitude from the rise position to the fall position.

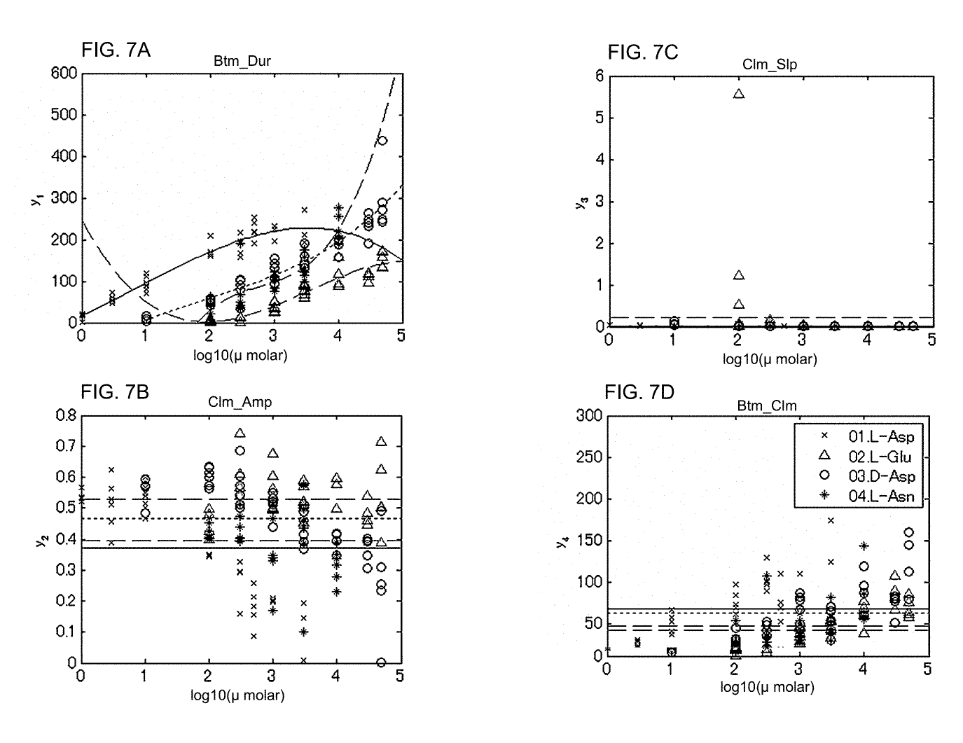

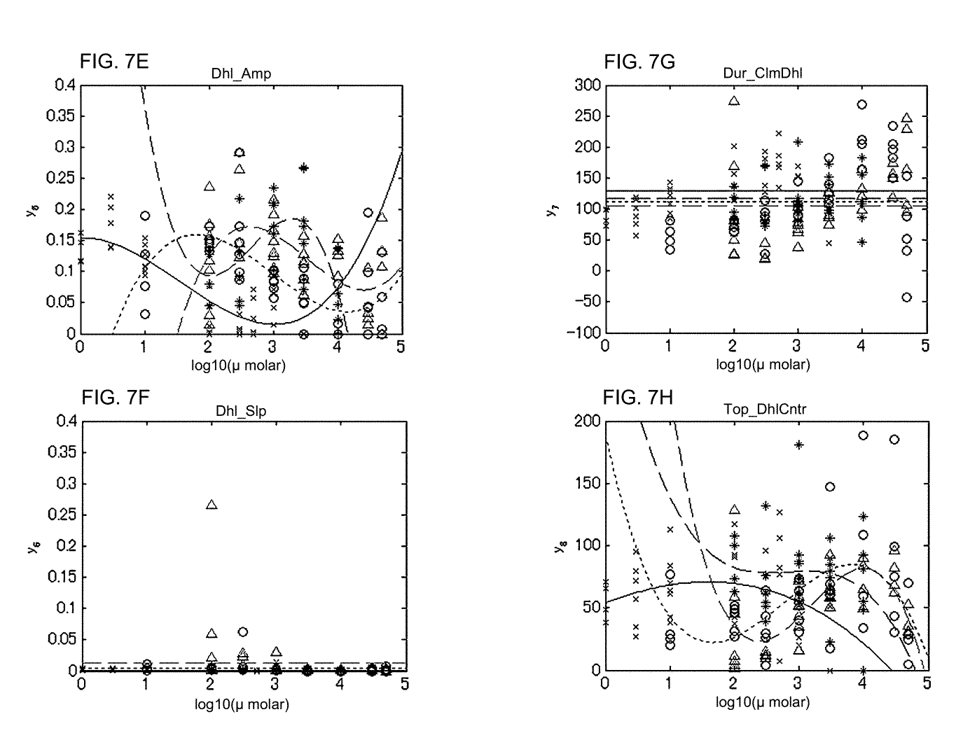

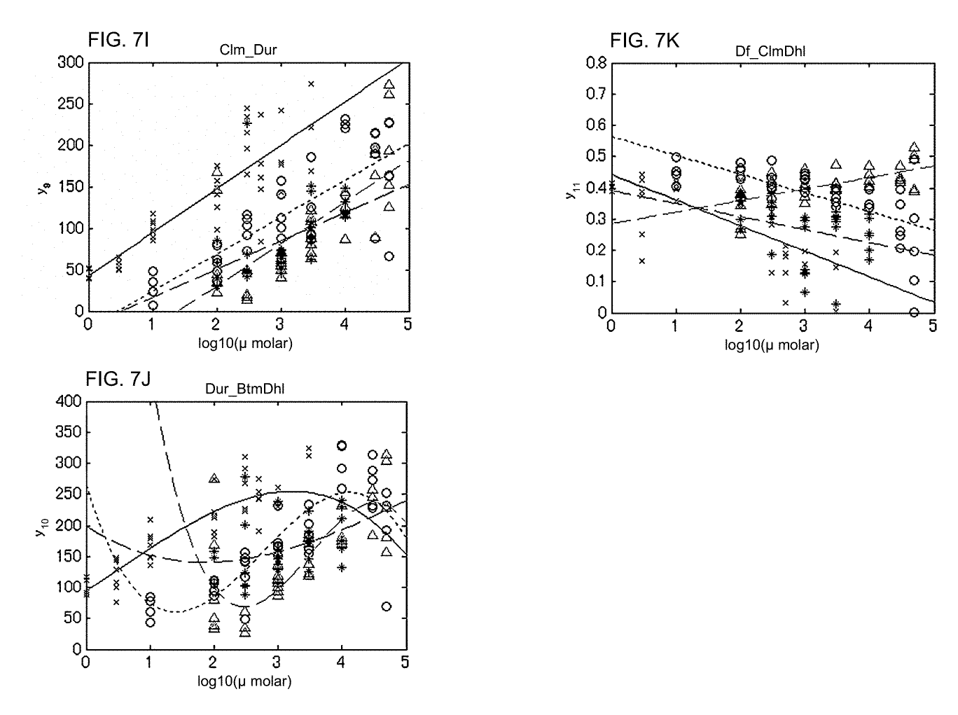

Next, using values extracted from the above-described 11 parameters, 11 graphs (i.e., models) showing these values on the vertical axis thereof, and logarithms of the sample concentration on the horizontal axis thereof are created. These 11 graphs are shown in FIG. 7. In FIG. 7, in sequence from the top of the left-hand column looking at the sheet are graphs whose vertical axes are set respectively to values based on the parameters of (1), values based on the parameters of (2), values based on the parameters of (3), values based on the parameters of (4), values based on the parameters of (5), values based on the parameters of (6), values based on the parameters of (7), values based on the parameters of (8), values based on the parameters of (9), values based on the parameters of (10), and values based on the parameters of (11).

Note that in the present embodiment, L-aspartic acid, L-glutamic acid, D-aspartic acid, and L-aspartic acid as represented by the following chemical formula were used respectively for the samples used to create FIG. 7.

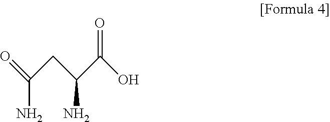

In the same way as for the sample data, the determination part 4 then represents the statistical data received from the statistical part 2 in a graph whose vertical axis shows the proportion of microorganisms that exhibit clockwise rotation behavior out of all of the E. coli bacteria contained within a unit area, and whose horizontal axis shows time, and then extracts the respective values based on the 11 parameters from this graph. The values based on the 11 parameters extracted from the statistical data are then adapted to the previously created 11 graphs (i.e., models), and the probability of a predetermined chemical substance, or alternatively the probability of a concentration of a predetermined chemical substance that the statistical data is able to generate is calculated, and the predetermined chemical substance used when the statistical data was acquired is estimated. This estimation of the chemical substance may be an estimation of the chemical substance or an estimation of the concentration of the chemical substance. The estimated contents are then output to an external device such as a display device or the like.

The sensing device 1 of the present embodiment which is formed in the above-described manner exhibits the following exceptional effects.

Namely, by acquiring for each chemical substance a plurality of sample data items created by compiling in advance in statistical form the behavior of a plurality of microorganisms that change over time in an environment in which a known chemical substance is present, and by using this sample data together with statistical data created by compiling in statistical form the behavior of a plurality of microorganisms that change over time in an environment in which a blind (unknown) chemical substance is present, it is possible to estimate the blind chemical substance. Moreover, even if the specialized senses of an organism are not able to be converted into physical quantities, by compiling in statistical form the behavior of an organism which is converted over time, that organism can be used as a biosensor.

Moreover, because the determination part 4 estimates a predetermined chemical substance using Bayesian inference, even if the model that is hypothesized based on the sample data is a complex one, operating the model can be simplified compared to when a maximum likelihood method is used.

Furthermore, because the statistical part 2 creates the statistical data using ensemble averages, it is possible to compile the behavior of the microorganisms in statistical form while considering irregularities and the like caused by individual differences in the microorganisms, so that the accuracy of the statistical data can be further improved.

Furthermore, because bacteria and, in particular, E. coli bacteria are used as the microorganisms, handling the microorganisms is easy, and the microorganisms can be easily cultured. As a consequence, statistical data can be easily acquired. Moreover, if the E. coli bacteria are genetically engineered so as to create bacteria whose taste senses prefer a specific chemical substance, then it is possible to increase the number of categories of chemical substances that can be measured.

The present invention is not limited to the above-described embodiment.

In the above-described embodiment, E. coli bacteria are used as the microorganisms; however, the present invention is not limited to this and provided that microorganisms are used, then any type of microorganism can be used. In particular, if bacteria are used, then because bacteria are easily handled, statistical data can be easily acquired.

The graph that is based on the 11 parameters created from the sample data by the determination part is not limited to these parameters, and the parameters can be altered in an appropriate manner. Moreover, in the present embodiment, the determination part estimates a predetermined chemical substance Bayesian inference, however, the present invention is not limited to Bayesian inference and the estimation method employed by the determination part may also be suitably altered. Because of this, it is also possible, for example, to directly compare the sample data and the statistical data, and to thereby determine the predetermined chemical substance that is used when the statistical data was acquired.

Moreover, in the above-described embodiment, ensemble averages are used when the statistical part creates the statistical data, however, it is also possible for the statistical data to be created using ensemble averages or using some other method.

It is also possible for various other modifications to be made to the present invention insofar as these are not in opposition to the spirit or scope thereof.

### INDUSTRIAL APPLICABILITY

According to the present invention, it is possible to provide a sensing device and the like that, by using the behavior of a plurality of microorganisms that change over time as a sensor, enable a chemical substance to be estimated.

What is claimed is:

1. A sensing device comprising:
a recording part in which are recorded a plurality of sample data items created by compiling in advance in statistical form the behavior of a plurality of microorganisms that change over time in an environment in which a known chemical substance is present;
a statistical part that creates statistical data by compiling in statistical form the behavior of the plurality of microorganisms that change over time in an environment in which a predetermined chemical substance is present; and
a determination part that, using the statistical data and the sample data, estimates the predetermined chemical substance.

2. The sensing device according to claim 1, wherein the determination part creates a model based on the sample data, and uses Bayesian inference to estimate the predetermined chemical substance by adapting the statistical data to the model.

3. The sensing device according to claim 1, wherein the statistical part creates the statistical data using ensemble averages.

4. The sensing device according to claim 1, wherein the microorganisms are bacteria, and the behavior of the bacteria is a rotational behavior.

5. A sensing method in which a predetermined chemical substance is estimated using sample data created by compiling in advance in statistical form the behavior of a plurality of microorganisms that change over time in an environment in which a known chemical substance is present, and statistical data created by compiling in statistical form the behavior of the plurality of microorganisms that change over time in an environment in which the predetermined chemical substance is present.

6. A sensing program that estimates a predetermined chemical substance using sample data created by compiling in advance in statistical form the behavior of a plurality of microorganisms that change over time in an environment in which a known chemical substance is present, and statistical data created by compiling in statistical form the behavior of the plurality of microorganisms that change over time in an environment in which the predetermined chemical substance is present.
